(19) United States
(12) Patent Application Publication
(10) Pub. No.: US 2009/0004670 A1
(43) Pub. Date: Jan. 1, 2009

(54) METHODS FOR FABRICATING SURFACE ENHANCED FLUORESCENT (SEF) NANOPARTICLES AND THEIR APPLICATIONS IN BIOASSAYS

(76) Inventors: Jingwu Zhang, San Jose, CA (US); David J. Liu, Fremont, CA (US)

Correspondence Address: Client 21058;c/o DARBY & DARBY P.C., P.O. BOX 770, CHURCH STREET STATION, NEW YORK, NY, 10008-0770, US

(21) Appl. No.: 11/771,675

(22) Filed: Jun. 29, 2007

(51) Int. Cl. B05D 5/06; G01N 33/533; G01N 33/53; B32B 5/16; C07K 17/00; C07K 16/00; C07H 21/00

(52) U.S. Cl. 435/7.1; 427/157; 436/518; 428/402; 428/403; 428/404; 530/391.3; 530/403; 536/23.1

(57) ABSTRACT

Embodiments of the invention relate to SEF nanoparticles with increased fluorescence, methods of making SEF nanoparticles, and their application in various bioassays for the detection of target bioanalytes. One embodiment includes the SEF nanoparticle itself, a second embodiment includes the fabrication of SEF nanoparticles, a third embodiment includes methods of using SEF nanoparticles in biodetection assays. A final embodiment includes kits to be used in the fabrication of SEF nanoparticles.

### FIELD OF INVENTION

Embodiments of the invention relate to surface-enhanced fluorescent (SEF) nanoparticles, methods for fabricating SEF nanoparticles, and methods of using SEF nanoparticles to detect target biological molecules with high sensitivity. The embodiments are especially directed to fabricating and utilizing SEF nanoparticles that demonstrate lower toxicity, higher signal intensity, and increased photostability. The invention transcends several scientific disciplines such as organic chemistry, polymer chemistry, surface chemistry, biochemistry, molecular biology, medicine and medical diagnostics.

### BACKGROUND

The ability to detect and identify trace quantities of analytes has become increasingly important in virtually every scientific discipline, ranging from part per billion analyses of pollutants in sub-surface water to analysis of cancer treatment drugs in blood serum.

With the advancement of technologies to make and detect biomolecules, there are multiple techniques that promise biological detection with single molecule sensitivity. However, many of these techniques have not yet found commercial applications. The main reasons are the complexity associated with these ultra-sensitive methods. Many art-known methods require multiple steps and chemical treatments, bulky and expensive instruments, and/or extreme care in sample handling and observation. These are not ideal for practical applications that require easy and reliable measurements or target biomolecule detection.

Nanotechnology is a term used to describe the fabrication, characteristics, and use of structures (“nanoparticles”) that have nanometer dimensions. Nanoparticles are so small that they exhibit quantum mechanical effects that allow them to interact strongly with light waves, even though the wavelength of the light may be much larger than the particle. Nanoparticles are frequently produced by chemical reactions in solutions. They are quite different from the same materials in bulky size, which do not exhibit quantum effects.

All objects with a metal surface, including nanoshells and metal core objects, exhibit a phenomenon called “surface plasmon resonance” in which incident light is converted strongly into electron currents at the metal surface. The oscillating currents produce strong electric fields in the (non-conducting) ambient medium near the surface of the metal. The electric fields, in turn, induce electric polarization in the ambient medium. Electric polarization is well known to cause the emission of light at wavelengths characteristic of the medium, the Raman wavelengths. Additional background information regarding this phenomenon may be found in Surface Enhanced Raman Scattering, ed. Chang & Furtak, Plenum Press, NY (1982), the entire disclosure of which is incorporated herein by reference. Other types of nanoparticles are known that are capable of stimulating surface enhanced Raman emissions from nearby materials, such as, gold clusters. In this application, the term Raman scattering is intended to encompass all related physical phenomena where the optical wave interacts with the polarizability of the material, such as Brillouin scattering or polariton scattering.

Detection and identification of the wavelengths of Raman emission can be used to “fingerprint” and locate the components of the ambient medium. The process of stimulating the surface plasmon resonance with light and subsequent emission of light at Raman wavelengths is called “surface enhanced Raman scattering” (SERS). The advantage of nanoparticles for SERS is the ability to tune the wavelength of the surface plasmon resonance to any desired value by adjusting the thickness of the shell and diameter of the dielectric sphere. For purposes of this invention, it may be desirable to tune the resonance to the near infrared, where transmission through optical fiber glass is possible over long distances with little absorption and where inexpensive laser sources exist.

SERS has been shown to enhance the intensity of Raman scattering in material near the surface of the metal surface by as much as 10¹⁴. In Surface enhanced Raman scattering in the near infrared using metal nanoshell substrates, S. J. Oldenburg, et. al., J. Chem. Phys. 111 (1999) 4729, for instance, nanoshells were suspended in a colloidal solution containing the organic compound p-mercaptoaniline and the Raman scattering intensity was compared to the same solution without suspended nanoshells. The p-mercaptoaniline Raman enhancement in this case was reported to be a factor of approximately 200,000.

Fluorescent nanoparticles have found a wide range of applications such as ultrasensitive detection of biomolecules, fluorescent imaging, flow cytometry, and high-throughput drug screening. Currently available fluorescent particles include quantum dots, dye-loaded latex spheres, dye-doped silica particles and π-conjugated polymer nanoparticles. These fluorescent particles have relatively high brightness and photostability as compared with dye molecules themselves. However, the toxicity of cadmium in quantum dots and relatively large size of dye-loaded particles have limited their applications. For example, dye-loaded latex spheres normally have large size in microns.

Although very small size (down to 10 nm in diameter) has been achieved for conjugated polymer particles, their signal intensity is lower than the larger fluorescent particles. Lower signal intensity makes the particles more difficult to detect with conventional techniques.

### BRIEF DESCRIPTION OF THE DRAWINGS

FIG. 1 is a schematic representation of the general structure of SEF nanoparticles.

FIG. 2 is a schematic representation of a SEF nanoparticle containing a single metal particle, or alternatively, a cluster of metal particles.

FIG. 3 is a schematic representation of the synthesis (i.e., fabrication) of SEF nanoparticles using silica as a spacer, matrix for the fluorophore zone, and an encapsulation layer.

FIG. 4 is a schematic representation of the synthesis (i.e., fabrication) of SEF nanoparticle clusters using conjugated dye polymers and encapsulation with silica.

FIG. 5 is a schematic representation of SEF nanoparticle bioconjugation to an affinity agent, and subsequent bioanalyte detection. Step A) shows the EDC-mediated coupling of an affinity agent that targets specific biomolecules to the outer surface of the SEF nanoparticle. Step B) shows the detection of target biomolecules through affinity capturing.

### DETAILED DESCRIPTION

As used in the specification and claims, the singular forms “a,” “an,” and “the” include plural references unless the context clearly dictates otherwise. For example, the term “an array” may include a plurality of arrays unless the context clearly dictates otherwise.

A “SEF nanoparticle” is one or more of an intentionally created surface-enhanced fluorescent nanoparticle that can be prepared either synthetically or biosynthetically. The SEF nanoparticles have one or more “core metal nanoparticles” that are coated with a fluorophore zone. Preferred core metal nanoparticles as used herein are metallic nanoparticles. The core metal nanoparticle can be any metal, including noble metals, and alloys. More preferred nanoparticles include coinage (Au, Ag, Cu), alkalis (Li, Na, K), Al, Pd, Ni, and Pt. A “SEF nanoparticle cluster” is a SEF nanoparticle that includes two or more core metal (or alloy) nanoparticles. SEF nanoparticles can have multiple layers, including but not limited to, one or more fluorophore zones, one or more spacer layers, and one or more encapsulation layers.

SEF nanoparticles, as described above, elicit plasmon resonance when excited with electromagnetic energy. A plasmon resonant particle can be “optically observable” when it exhibits significant scattering intensity in the optical region, which includes wavelengths from approximately 180 nanometers (nm) to several microns. A SEF nanoparticles can be “visually observable” when it exhibits significant scattering intensity in the wavelength band from approximately 400 nm to 700 nm which is detectable by the human eye. Plasmon resonance is created via the interaction of incident light with basically free conduction electrons. The particles or entities have dimensions, e.g., diameters preferably about 25 to 150 nm, more preferably, about 40 to 100 nm.

The term “plasmon resonant entity” or “PRE” refers to any independent structure exhibiting plasmon resonance characteristic of the structure, including (but not limited to) both SEF nanoparticles and combinations or associations of SEF nanoparticles as defined and described above. A SEF nanoparticle may include either a single SEF nanoparticle or an aggregate of two or more SEF nanoparticles that manifest a plasmon resonance characteristic when excited with electromagnetic energy.

“Plasmon absorption” is the extinction of light (by absorption and scattering) caused by the metal surface plasmons which are quantified and localized oscillations of electron density in metals.

The terms “nanomaterial” and “nanoparticle” as used herein refer to a structure, a device, or a system having a dimension at the atomic, molecular or macromolecular levels, in the length scale of approximately 1-1000 nanometer range, preferably in the range of about 2 nm to about 200 nm, more preferably in the range of about 2 nm to about 100 nm.

The term “SEF nanoparticle” as used herein refers to the modified nanoparticles of the invention, while the term “nanoparticle,” without qualification, refers to a nanoparticle that serves as the inner core of the SEF nanoparticle. Preferably, a nanomaterial has properties and functions because of the size and can be manipulated and controlled on the atomic level. Nanoparticles made of semiconductor material may also be labeled quantum dots if they are small enough (typically sub 10 nm) that quantization of electronic energy levels occurs.

The term “core metal nanoparticle refers to a nanoparticle as defined above that is composed of a metallic material, an alloy, or other mixture of metallic materials, or a metallic core contained within one or more metallic overcoat layers.

The term “analyte,” “bioanalyte,” “target,” or “target molecule” refers to a molecule of interest that is to be analyzed, detected, and/or quantified in some manner. The analyte may be a biological species, including, but not limited to, nucleic acids, proteins, toxins, pathogens, bacterium cells, virus cells, cancer cells, normal cells, organisms, tissues. The analyte may be a Raman active compound or a Raman inactive compound. Further, the analyte could be an organic or inorganic molecule. Some examples of analytes may include a small molecule, a biomolecule, or a nanomaterial such as but not necessarily limited to a small molecule that is biologically active, nucleic acids and their sequences, peptides and polypeptides, as well as nanostructure materials chemically modified with biomolecules or small molecules capable of binding to molecular probes such as chemically modified carbon nanotubes, carbon nanotube bundles, nanowires, nanoclusters or nanoparticles. The analyte molecule may be a fluorescently labeled molecule, such as for example, DNA, RNA or protein. Disease cells refer to cells that would be considered pathological by a person of ordinary skill in the art, such as a pathologist. Non-limiting examples of disease cells include tumor cells, gangrenous cells, virally or bacterially infected cells, and metabolically aberrant cells.

The term “bi-functional linker group” refers to an organic chemical compound that has at least two chemical groups or moieties, such as for example, carboxyl group, amine group, thiol group, aldehyde group, epoxy group, that can be covalently modified specifically; the distance between these groups is equal to or greater than 5-carbon bonds.

The term “affinity agent” or “capture molecule” refers to a molecule that is bound (“bioconjugated”), reversibly or irreversibly, to a SEF nanoparticle. The capture molecule generally, but not necessarily, binds to one or more targets or target molecules, as described above. The capture molecule is typically an antibody, an aptamer, an oligonucleotide, or a protein, but could also be a small molecule, biomolecule, or nanomaterial such as, but not necessarily limited to, a small molecule that is biologically active, nucleic acids and their sequences, peptides and polypeptides, as well as nanostructure materials chemically modified with biomolecules or small molecules capable of binding to a target molecule that is bound to a probe molecule to form a complex of the capture molecule, target molecule and the probe molecule. The capture molecule may be fluorescently labeled DNA or RNA. The capture molecule may or may not be capable of binding to just the target molecule or just the probe molecule. Other capture molecules include, for example, antibody fragments, antigens, epitopes, lectins, sialic acid and other carbohydrates, proteins, polypeptides, receptor proteins, ligands, hormones, vitamins, metabolites, substrates, inhibitors, cofactors, pharmaceuticals, cytokines and neurotransmitters

The term “molecule” generally refers to a macromolecule or polymer as described herein. However, SEF nanoparticles comprising single molecules, as opposed to macromolecules or polymers, are also within the scope of the embodiments of the invention.

A “macromolecule” or “polymer” comprises two or more monomers covalently joined. The monomers may be joined one at a time or in strings of multiple monomers, ordinarily known as “oligomers.” Thus, for example, one monomer and a string of five monomers may be joined to form a macromolecule (polymer) of six monomers. Similarly, a string of fifty monomers may be joined with a string of hundred monomers to form a macromolecule or polymer of one hundred and fifty monomers. The term polymer as used herein includes, for example, both linear and cyclic polymers of nucleic acids, polynucleotides, polynucleotides, polysaccharides, oligosaccharides, proteins, polypeptides, peptides, phospholipids and peptide nucleic acids (PNAs). The peptides include those peptides having either α-, β-, or ω-amino acids. In addition, polymers include heteropolymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which will be apparent upon review of this disclosure.

The term “nucleotide” includes deoxynucleotides and analogs thereof. These analogs are those molecules having some structural features in common with a naturally occurring nucleotide such that when incorporated into a polynucleotide sequence, they allow hybridization with a complementary polynucleotide in solution. Typically, these analogs are derived from naturally occurring nucleotides by replacing and/or modifying the base, the ribose or the phosphodiester moiety. The changes can be tailor-made to stabilize or destabilize hybrid formation, or to enhance the specificity of hybridization with a complementary polynucleotide sequence as desired, or to enhance stability of the polynucleotide.

The term “polynucleotide” or “nucleic acid” as used herein refers to a polymeric form of nucleotides of any length, either ribonucleotides or deoxyribonucleotides, that comprise purine and pyrimidine bases, or other natural, chemically or biochemically modified, non-natural, or derivatized nucleotide bases. Polynucleotides of the embodiments of the invention include sequences of deoxyribopolynucleotide (DNA), ribopolynucleotide (RNA), or DNA copies of ribopolynucleotide (cDNA) which may be isolated from natural sources, recombinantly produced, or artificially synthesized. A further example of a polynucleotide of the embodiments of the invention may be polyamide polynucleotide (PNA). The polynucleotides and nucleic acids may exist as single-stranded or double-stranded. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The polymers made of nucleotides such as nucleic acids, polynucleotides and polynucleotides may also be referred to herein as “nucleotide polymers.

An “oligonucleotide” is a polynucleotide having 2 to 20 nucleotides. Analogs also include protected and/or modified monomers as are conventionally used in polynucleotide synthesis. As one of skill in the art is well aware, polynucleotide synthesis uses a variety of base-protected nucleoside derivatives in which one or more of the nitrogen atoms of the purine and pyrimidine moiety are protected by groups such as dimethoxytrityl, benzyl, tert-butyl, isobutyl and the like.

For instance, structural groups are optionally added to the ribose or base of a nucleoside for incorporation into a polynucleotide, such as a methyl, propyl or allyl group at the 2′-O position on the ribose, or a fluoro group which substitutes for the 2′-O group, or a bromo group on the ribonucleoside base. 2′-O-methyloligoribonucleotides (2′-O-MeORNs) have a higher affinity for complementary polynucleotides (especially RNA) than their unmodified counterparts. Alternatively, deazapurines and deazapyrimidines in which one or more N atoms of the purine or pyrimidine heterocyclic ring are replaced by C atoms can also be used.

The phosphodiester linkage, or “sugar-phosphate backbone” of the polynucleotide can also be substituted or modified, for instance with methyl phosphonates, O-methyl phosphates or phosphororthioates. Another example of a polynucleotide comprising such modified linkages for purposes of this disclosure includes “peptide polynucleotides” in which a polyamide backbone is attached to polynucleotide bases, or modified polynucleotide bases. Peptide polynucleotides which comprise a polyamide backbone and the bases found in naturally occurring nucleotides are commercially available.

When the macromolecule of interest is a peptide, the amino acids can be any amino acids, including α, β, or ω-amino acids. When the amino acids are α-amino acids, either the L-optical isomer or the D-optical isomer may be used. Additionally, unnatural amino acids, for example, β-alanine, phenylglycine and homoarginine are also contemplated by the embodiments of the invention. These amino acids are well-known in the art.

A “peptide” is a polymer in which the monomers are amino acids and which are joined together through amide bonds and alternatively referred to as a polypeptide. In the context of this specification it should be appreciated that the amino acids may be the L-optical isomer or the D-optical isomer. Peptides are two or more amino acid monomers long, and often more than 20 amino acid monomers long.

A “protein” is a long polymer of amino acids linked via peptide bonds and which may be composed of two or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, and antibodies.

The term “sequence” refers to the particular ordering of monomers within a macromolecule and it may be referred to herein as the sequence of the macromolecule.

A “ligand” is a molecule that is recognized by a particular receptor. Examples of ligands that can be investigated by this invention include, but are not restricted to, agonists and antagonists for cell membrane receptors, toxins and venoms, viral epitopes, hormones, hormone receptors, peptides, enzymes, enzyme substrates, cofactors, drugs (e.g. opiates, steroids, etc.), lectins, sugars, polynucleotides, nucleic acids, oligosaccharides, proteins, and monoclonal antibodies.

A “receptor” is molecule that has an affinity for a given ligand. Receptors may-be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term “receptors” is used herein, no difference in meaning is intended. A “Ligand-Receptor Pair” is formed when two macromolecules have combined through molecular recognition to form a complex. Other examples of receptors which can be investigated by this invention include but are not restricted to:

a) Microorganism receptors: Determination of ligands which bind to receptors, such as specific transport proteins or enzymes essential to survival of microorganisms, is useful in developing a new class of antibiotics. Of particular value would be antibiotics against opportunistic fungi, protozoa, and those bacteria resistant to the antibiotics in current use.

b) Enzymes: For instance, one type of receptor is the binding site of enzymes such as the enzymes responsible for cleaving neurotransmitters; determination of ligands which bind to certain receptors to modulate the action of the enzymes which cleave the different neurotransmitters is useful in the development of drugs which can be used in the treatment of disorders of neurotransmission.

c) Antibodies (Abs): For instance, the invention may be useful in investigating the ligand-binding site on the antibody molecule which combines with the epitope of an antigen of interest; determining a sequence that mimics an antigenic epitope may lead to the development of vaccines of which the immunogen is based on one or more of such sequences or lead to the development of related diagnostic agents or compounds useful in therapeutic treatments such as for auto-immune diseases (e.g., by blocking the binding of the “anti-self” antibodies). There are monoclonal antibodies (mAb) and polyclonal antibodies (pAb).

d) Nucleic Acids: Sequences of nucleic acids may be synthesized to establish DNA or RNA binding sequences. Certain sequence of nucleic acids, called aptamer, can bind to proteins or peptides.

e) Catalytic Polypeptides: Polymers, preferably polypeptides, which are capable of promoting a chemical reaction involving the conversion of one or more reactants to one or more products. Such polypeptides generally include a binding site specific for at least one reactant or reaction intermediate and an active functionality proximate to the binding site, which functionality is capable of chemically modifying the bound reactant.

f) Hormone receptors: Examples of hormones receptors include, e.g., the receptors for insulin and growth hormone. Determination of the ligands which bind with high affinity to a receptor is useful in the development of, for example, an oral replacement of the daily injections which diabetics take to relieve the symptoms of diabetes. Other examples are the vasoconstrictive hormone receptors; determination of those ligands which bind to a receptor may lead to the development of drugs to control blood pressure.

g) Opiate receptors: Determination of ligands which bind to the opiate receptors in the brain is useful in the development of less-addictive replacements for morphine and related drugs.

A “linker” molecule refers to any of those molecules described supra and preferably should be about 4 to about 100 atoms long to provide sufficient exposure. The linker molecules may be, for example, aryl acetylene, alkane derivatives, ethylene glycol oligomers containing 2-10 monomer units, diamines, diacids, amino acids, among others, and combinations thereof. Alternatively, the linkers may be the same molecule type as that being synthesized (i.e., nascent polymers), such as polynucleotides, oligopeptides, or oligosaccharides.

The term “fluid” used herein means an aggregate of matter that has the tendency to assume the shape of its container, for example a liquid or gas. Analytes in fluid form can include fluid suspensions and solutions of solid particle analytes.

The term “fluorophore” means a component of a molecule or substance which causes a molecule or substance to be fluorescent. It is a functional group in a molecule which will absorb energy of a specific wavelength, and re-emit energy at a different (but equally specific) wavelength. The intensity and wavelength of the emitted energy depend on both the fluorophore and the chemical environment of the fluorophore. Fluorophores have particular importance in the field of biochemistry and protein studies, eg. in immunofluorescence and immunohistochemistry.

The term “attached,” as in, for example, the “attachment” of an affinity agent to a SEF nanoparticle surface, includes covalent binding, adsorption, and physical immobilization. The terms “associated with,” “binding” and “bound” are identical in meaning to the term “attached.”

The term “TEOS” refers to for tetraethylorthosilicate.

The “Stöber process” is a method to prepare silica particles by hydrolysis of TEOS in presence of ammonium hydroxide in organic solvent (such as methanol, ethanol, n-propanol, n-butanol). The original method is described by the 1968 paper of W. Stöber, A. Fink and E. Bohn, J. Colloid Int. Sci 26:62-69 (1968) (disclosure of which is hereby incorporated by reference). Various modifications (in terms of reagent concentration, water content, reaction time) have been made by different authors to provide a silica coating on different particles.

Embodiments of the invention relate generally to SEF nanoparticles, methods of fabricating SEF nanoparticles, methods of detecting target bioanalytes using SEF nanoparticle probes, and kits (such as for using in the laboratory setting) containing the reagents necessary to make, synthesize, and/or use desired SEF nanoparticles, depending on the user's planned application. The methods and products allow the fabrication of SEF nanoparticles, and their use in the detection of biological molecules with ultra-sensitivity and convenience. The embodiments are especially directed to utilizing SEF nanoparticles as “tags,” and identifying the tags using fluorescence microscopy or other detection methods wherein the fluorescent SEF nanoparticles can be detected and/or observed. Probes containing the SEF nanoparticles can be used in solution or attached to a substrate, depending on user needs.

One embodiment is a method of fabricating a surface enhanced fluorescent (SEF) nanoparticle. The method includes obtaining one or more core metal nanoparticles having desired plasmonic properties, optionally coating the core metal nanoparticle with a spacer layer; optionally adding a primer zone to the spacer layer; adding a fluorophore zone; and optionally adding an encapsulation layer. In certain embodiments, SEF nanoparticles have only a core metal nanoparticle coated with a fluorophore zone.

The core nanoparticle is a metal or alloy, preferably a noble metal, or alloy thereof. Preferred metals include Au, Ag, Cu, Al, Pd and Pt. More preferably, the core metal nanoparticle is Ag or Au or their alloy. In certain embodiments, the metal is aluminum or an alloy thereof. The core metal can be selected based on the needs of a user.

The optional spacer layer is silica or an organic polymer, preferably silica.

The SEF nanoparticle includes a fluorophore zone that is comprised of one or more fluorophores.

Preferably the optional encapsulation layer comprises silica or an organic layer.

The SEF nanoparticle includes either 1 core metal nanoparticle, or optionally, more than 1 core metal nanoparticle (i.e., a “cluster”).

Another embodiment is a method of detecting a target analyte, such as a biomolecule of interest, with a SEF nanoparticle by attaching (“bioconjugating”) an affinity agent to one or more SEF nanoparticles; contacting the bioconjugated SEF nanoparticle with at least one target analyte of interest; and detecting the bioconjugated SEF nanoparticle.

The affinity agent is preferably an antibody, antigen, ligand, receptor, aptamer, or a nucleic acid. More preferably, the affinity agent is an antibody or a nucleic acid.

The target analyte is a biomolecule. Preferably, the target analyte is a nucleic acid or a protein of interest.

Another embodiment of the invention is a SEF nanoparticle having one or more core metal nanoparticles and a fluorophore zone. The SEF nanoparticle may also optionally have a spacer layer located between the core metal nanoparticles and the fluorophore zone. Preferably, the spacer layer has an outer primer layer. The SEF nanoparticle may also have an encapsulation layer surrounding (i.e., as the outermost layer of) the SEF nanoparticle.

Preferably the one or more core nanoparticle(s) are a metal. More preferably, the core metal nanoparticle is a noble metal. Optionally, the core metal nanoparticle is an alloy. The noble metal is preferably Au, Ag, Cu, Al, Pd, Pt, or alloys thereof. More preferably, the noble metal is Ag or Au or their alloy.

The SEF nanoparticle includes a fluorophore zone that is comprised of one or more fluorophores. Preferred fluorophores include those that fluoresce under light wavelengths from about 400 to about 700 nm.

The fluorophore zone is preferably one layer with a thickness of less than about 20 nm. Within this layer, different types of fluorophores which are known in the art can be incorporated. Preferably, materials for the fluorophore zone are transparent to both incident and emitting light. Organic polymer matrix can be used to incorporate the fluorescent molecules. Preferable organic polymers include, for example, polystyrene, polyacrylamide, polyacrylic acid, polyolefin, polyvinylpyridine, etc.

Optionally, the SEF nanoparticle may include a spacer layer. Preferably, the spacer layer is silica or an organic polymer, more preferably silica. Other materials for the spacer layer include electronically insulating materials that are transparent to the incident and emitting light, such as for example, calcium phosphates, iron oxide, titanium oxide, organic polymers (both synthetic and natural), biopolymers such as bovine serum albumin.

The thinnest spacer layer is made of a monolayer of molecules. There is no absolute upper limit for the space layer thickness. However, as the surface enhancement factor generally decreases with increasing the distance from the surface, the upper limit for the thickness of the spacer layer is practically about 10 nm.

Preferably, the optional encapsulation layer includes silica or an organic layer. The encapsulation layer is the outmost layer of the SEF nanoparticles. Preferably, the encapsulation layer serves the following functions: (1) to prevent leakage of the fluorescent dyes from the fluorophore zone and thus to enhance photostability; (2) to provide surface attachment area for functional groups, such as for example affinity agents, for various applications; (3) to provide colloidal stability of the SEF nanoparticle in the suspension media and application media. However, none of these functions are required. The thickness of the encapsulation layer depends on the materials used for the encapsulation layer, but preferably is from 1 to 20 nm.

The SEF nanoparticle includes either one core metal nanoparticle, or optionally, more than one core metal nanoparticle.

Preferably, the SEF nanoparticle also includes an affinity agent attached to the SEF nanoparticle, thereby forming a bioconjugate. Preferably, the affinity agent is an antibody, antigen, ligand, receptor, aptamer, or a nucleic acid. More preferably, the affinity agent is an antibody or a nucleic acid. In various embodiments, more than one, or more than one type of, affinity agent is attached to the SEF nanoparticle, depending on use needs.

The affinity agent can be attached (bound) to SEF nanoparticle (covalently or non-covalently). Preferably, the resultant bioconjugate (SEF nanoparticle+affinity agent) is used to detect biomolecules of interest. More than one affinity agent, or more than one type of affinity agent, may be bound to a SEF nanoparticle.

The present invention also embodies kits for manufacturing (i.e., fabricating) SEF nanoparticles. Preferably, a kit contains reagents having one or more core metal nanoparticles, reagents having fluorophores of the present invention, and the kit may also contain reagent(s) for optionally adding a spacer layer, primer layer, and encapsulation layer. A kit may further include reagents for attaching affinity agents to the manufactured SEF nanoparticle, the affinity agent is capable of binding or interacting with a biomolecule of interest, such as for example, binding to a nucleic acid, protein, or antibody of the present invention. Preferably, the affinity agent is an antibody or a nucleic acid. SEF nanoparticles are useful in diagnostics and cellular labeling. For example, SEF nanoparticles can be used to selectively label tumor cells, which can then be studied either by flow cytometry or by cell imaging. Preferably, such embodiments are used for clinical and therapeutic advantages in human subjects where evaluation and treatment of disease conditions (e.g., cancer and cancer therapy) can be utilized.

The reagent(s) of the kit can be provided as a liquid solution, attached to a solid support or as a dried powder. Preferably, when the reagent(s) are provided in a liquid solution, the liquid solution is an aqueous solution. Preferably, when the reagent(s) provided are attached to a solid support, the solid support can be chromatograph media, a test plate having a plurality of wells, or a microscope slide. When the reagent(s) provided are a dry powder, the powder can be reconstituted by the addition of a suitable solvent known in the art that may be provided.

It has been determined that surface-enhanced Raman scattering (SERS) can be used to detect the binding of one analyte to another. In particular, it has been found that the binding of small molecules (molecular weight less than 5,000 Da) to biomolecules, in particular proteins (such as enzymes) can be detected by SERS. This provides a new tool for interrogating small molecule-protein interaction.

The invention includes surface enhanced fluorescent nanoparticles, methods for fabricating such particles based on plasmonic enhancement. Their application in sensitive detection of biomolecules, and kits for the fabrication and use of surface enhanced nanoparticles. The novelty of this invention is that a noble metal core nanoparticle, or clusters of such noble metal core nanoparticles, are included in the fluorescent particles thereby increasing the fluorescence signal by several fold. Additionally, surface-enhanced fluorescent (SEF) nanoparticles with higher signal intensity (and thus easier detection) can be easily bioconjugated to affinity agents for sensitive biomolecule detection in various biological, clinical and diagnostic applications. The relatively high intensity, and the relatively small size of the SEF nanoparticles, make many new applications possible.

Embodiments of the invention include methods to fabricate SEF nanoparticles. To manufacture SEF nanoparticles, monodisperse noble metal particles or alloy particles with desired plasmonic properties are obtained. The core metal nanoparticles are optionally coated with a space layer comprised of silica or polymer to minimize quenching of fluorescence by the metal particle surface. A layer of one or more fluorophores, or a porous matrix layer which entraps fluorophores, is added to provide signal for detection. Optional encapsulation of SEF nanoparticles with silica or an organic layer to prevent leaching of dyes and fluorescence decay is envisioned. The encapsulation layer also provides functional groups for surface-modification, for example. The bioconjugation with affinity agents for the detection and analysis of desired biomolecules.

After manufacture of SEF nanoparticles, they can be bioconjugated with various affinity agents. This allows bioconjugated SEF nanoparticles to be used in desired applications, such as for example, targeting, quantifying, locating, and analyzing biomolecules such as, for example, nucleic acids and the like.

Embodiments of this invention have several useful applications. For example, SEF nanoparticles can be employed for the ultra-sensitive detection of bioanalytes including, antibodies, antigens, biomarkers, allergens, ligands, metabolites, virus, bacteria, tumor cells, etc. The ability to detect, locate, and/or quantify bioanalytes allows for diagnostic use, treatment, and/or monitoring of specific diseases, physiological conditions (normal or abnormal), conditions, and therapies. For example, abnormal proteins in human disease could be detected. As another example, the normal signal transduction inside, or outside cells could be detected and monitored. It is envisioned that SEF nanoparticles can be used to label cells selectively such that a specific type of cells can be studied either by flow cytometry or by cellular imaging. It is also envisioned that embodiments of the invention could be used in vivo or in vitro for screening purposes, i.e., high throughput methods of evaluating pathological conditions. High-throughput drug discovery screening is another example where embodiments of the invention would be useful.

Fluorescent imaging (e.g., at the cellular, tissue, and whole animal level) could be employed in both normal physiological systems, and also in pathological states for disease evaluation. Embodiments of the invention are also useful in flow cytometry, environmental monitoring, and food analysis.

Selective labeling of tumor cells or other disease cells with SEF nanoparticles can provide new tools for cancer and other disease diagnostics. The method for diagnostic use of SEF nanoparticles can be based either on flow cytometry or on fluorescent imaging.

In order to provide users with the ability to efficiently utilize embodiments of the invention, the present invention contemplates methods and kits for screening samples containing suspected analytes of interest that could be detected with SEF nanoparticles. The kits contain the reagents necessary to manufacture SEF nanoparticles, including core metal nanoparticles, reagents for adding an optional spacer and primer, the fluorophore zone, and encapsulation layer. The kit may optionally contain a variety of affinity agents that can be bioconjugated to the SEF nanoparticles and used in specific applications, such as for example, locating, quantifying, and or analyzing particular target biomolecules of interest.

For example, one kit contains all the reagents necessary for the production of SEF nanoparticles, and an affinity agent, such as a particular receptor, that is conjugated to the SEF nanoparticle. The particular receptor, when contacted to a sample of interest, will bind to a cellular protein of interest. The target sample can be derived from, for example, a cell culture (i.e., in vitro), or from a mammalian sample (i.e., in vivo). After contacting the SEF nanoparticle with bioconjugated receptor, and binding to biomolecule of interest, the SEF nanoparticle is detected, thereby detecting the presence (or absence), quantity, and location of the target cellular protein of interest. This example is merely illustrative, and not intended to be limiting.

Although embodiments have been described in which small molecules and proteins are described as being the analytes, it is understood, however, that the same process and tools can be used to detect the binding of a variety of analytes to one another and the invention is not limited to just the binding of small molecules to proteins.

### EXAMPLE 1

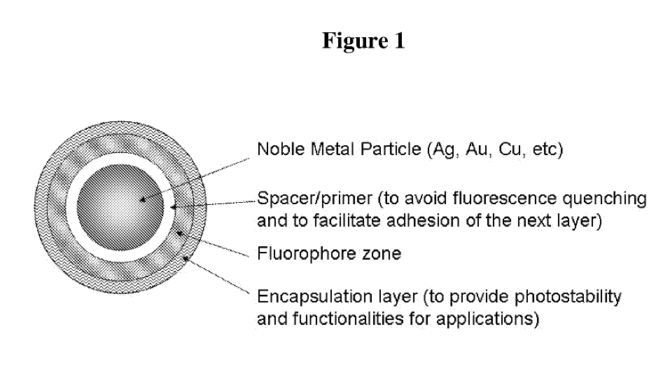

FIG. 1 shows an exemplary SEF nanoparticle. The SEF nanoparticle includes a core metal nanoparticle which can be a noble metal, such as for example, gold, copper or alloys thereof, that have the plasmon absorption at a desired wavelength. The core metal nanoparticle surface is coated with a spacer layer that prevents direct contact of fluorescent molecules (in the fluorophore zone) with the surface of the core metal particle, thus avoiding fluorescent quenching. The thickness of the spacer layer is about 3-5 nm, although thinner or thicker spacer layers are envisioned depending on the desired application. The spacer layer is made from silica, organic polymers, or other materials with low fluorescence background. A thin primer layer may optimally be added to (coated on) the spacer layer to facilitate adhesion, adsorption, or chemical bonding of the fluorophore zone. The fluorophore zone consists of a single layer, or multi-layers, of fluorescence molecules (including conjugated polymers). The fluorophore zone can be made from porous silica or an organic polymer matrix containing fluorophores. Typical organic polymers include, but are not limited to, polystyrene, polyacrylamide, polyacrylic acid, polyolefin, and polyvinylpyridine.

Finally, an encapsulation layer may be added to (coated on) the fluorophore zone to prevent leaching of dye molecules, to provide additional photostability, and to provide a substrate for the bioconjugation of functional groups such as affinity agents. The structure of SEF nanoparticles can be varied depending on the nature of fluorophores and desired applications.

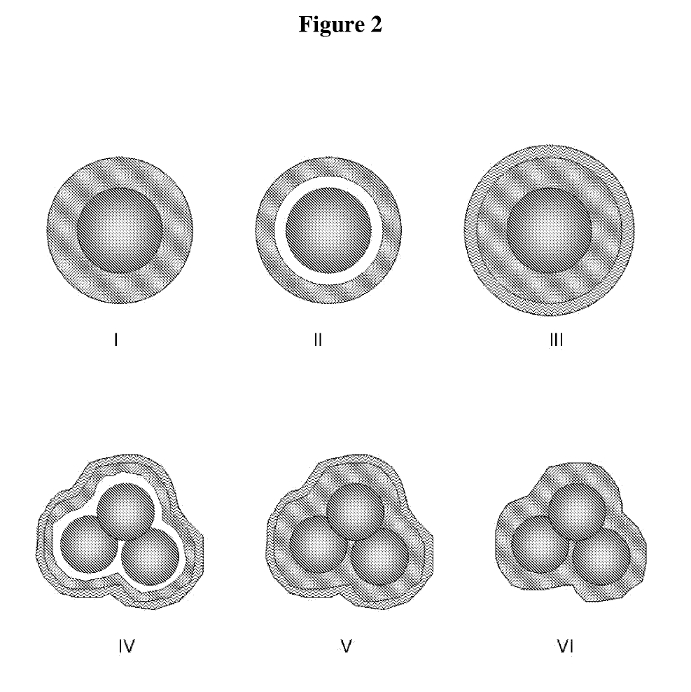

FIG. 2 demonstrates SEF nanoparticles wherein metal particle clusters are used as the core to enhance the fluorescence signals. Also noteworthy in FIG. 2 is the possibility of eliminating the spacer layer, i.e., the spacer layer is optional in all embodiments. Although some quenching occurs with the fluorophore in contact with a metal surface, and enhancement is expected to be lower when a fluorophore is in close proximity to a metal surface, fluorescence enhancement is expected for those fluorophores with certain distance from the surface. In addition, more fluorescent molecules can be included into the SEF particles when the spacer layer is removed. This can lead to a greater overall fluorescent signal. SEF nanoparticles having more than 1 core metal nanoparticle can have, for example two or more core metal nanoparticles. Such groups of core metal nanoparticles are called “clusters.” Cluster nanoparticles can also be bioconjugated with affinity agents, such as for example receptors, and contacted with samples for the detection of target biomolecules. By placing the fluorophore zone on or near a metallic core, Raman signal is significantly enhanced. It is thus possible to detect the SEF nanoparticles by using a Raman microscope in addition to fluorescent measurements. Therefore both Raman scattering and fluorescence emission can be determined at the same time, thereby confirming the detection of target bioanalytes when the SEF nanoparticle binds to the target bioanalyte through an affinity agent bound to the SEF nanoparticle.

### EXAMPLE 2

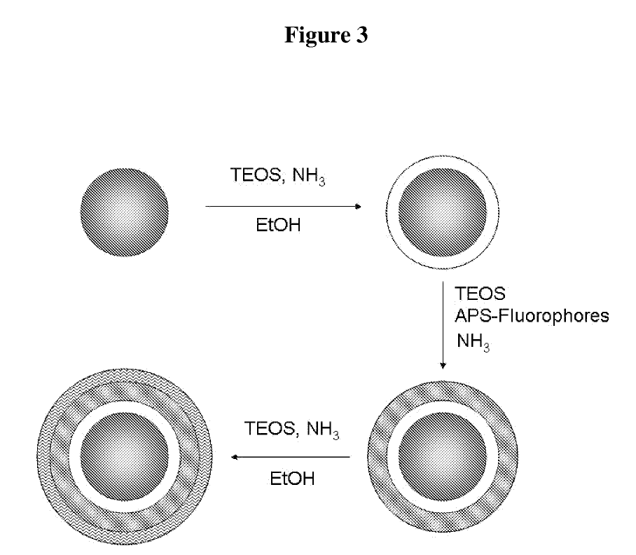

FIG. 3 illustrates a fabrication method of SEF nanoparticles by coating core metal particles with consecutive layers of silica. An optional spacer layer is generated by hydrolysis of TEOS in alcohol as used in the Stöber process. The fluorophore zone is subsequently added, in this case on top of an optional spacer layer, by simultaneous hydrolysis of TEOS and silane-modified fluorophores. The fluorophore zone is prepared by silica deposition in the presence of fluorophores. The optional encapsulation layer is then added by a modified Stöber process. Optional addition (bioconjugation) of functional groups, such as affinity agents, can be added to the encapsulation layer by silane chemistry (not shown). SEF nanoparticles can also be fabricated similarly with organic polymers used as the spacer layer, and organic matrix for the fluorophore zone and the encapsulation layer. Silica and polymer layers may also be used interchangeably to construct the SEF nanoparticles. As noted in Example 1 above, multiple (i.e., two or more) core metal nanoparticles may be used in this method, thereby creating “cluster” SEF nanoparticles.

### EXAMPLE 4

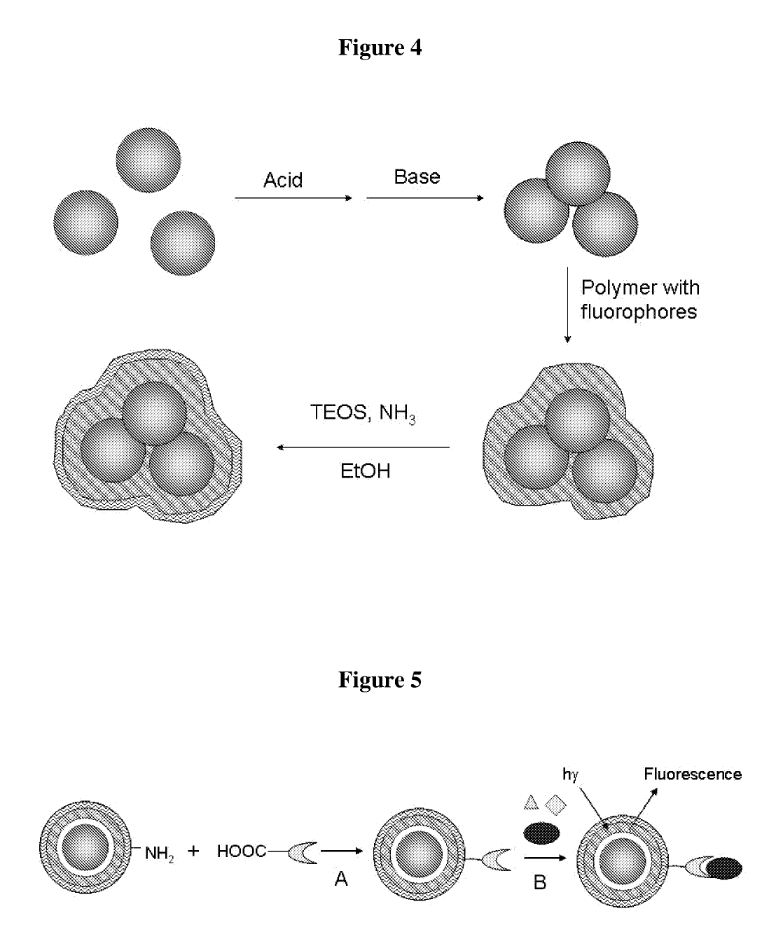

FIG. 4 shows a method of fabricating metal nanoclusters of controlled size by adjusting the pH of the suspending medium. Fluorescent polymers (with conjugated π-structure), or fluorophores grafted to a polymer backbone, are used to generate the fluorophore zone. Then, the structure can be encapsulated with a layer of silica or organic polymers.

### EXAMPLE 5

FIG. 5 demonstrates the outer surface bioconjugation of a SEF nanoparticle, and the use of such bioconjugated SEF nanoparticles for biomolecule detection. The SEF nanoparticles are functionalized with an affinity agent, such as an amine group. Various biomolecules of interest in a sample can be conjugated to the functionalized SEF nanoparticles through methods for bioconjugation that are well known in the art. Biomolecules of interest to be detected include, for example, proteins, antibodies, enzymes, nucleic acids (DNA, RNA, oligonucleotides), antigen, peptides, ligands, receptors, small molecules, metabolites, etc. Although the biological application of this SEF nanoparticle bioconjugates is immense, detection of signature antibody, autoantibody, antigen, virus and bacterium are of special interest for disease diagnostics and treatment monitoring.

Commercial applications for the products and methods described herein include environmental toxicology and remediation, biomedicine, materials quality control, food and agricultural products monitoring, anesthetic detection, automobile oil or radiator fluid monitoring, breath alcohol analyzers, hazardous spill identification, explosives detection, fugitive emission identification, medical diagnostics, detection and classification of bacteria and microorganisms both in vitro and in vivo for biomedical uses and medical diagnostic uses, monitoring heavy industrial manufacturing, ambient air monitoring, worker protection, emissions control, product quality testing, leak detection and identification, oil/gas petrochemical applications, combustible gas detection, H₂S monitoring, hazardous leak detection and identification, emergency response and law enforcement applications, illegal substance detection and identification, arson investigation, enclosed space surveying, utility and power applications, emissions monitoring, transformer fault detection, food/beverage/agriculture applications, freshness detection, fruit ripening control, fermentation process monitoring and control applications, flavor composition and identification, product quality and identification, refrigerant and fumigant detection, cosmetic/perfume/fragrance formulation, product quality testing, personal identification, chemical/plastics/pharmaceutical applications, leak detection, solvent recovery effectiveness, perimeter monitoring, product quality testing, hazardous waste site applications, fugitive emission detection and identification, leak detection and identification, perimeter monitoring, transportation, hazardous spill monitoring, refueling operations, shipping container inspection, diesel/gasoline/aviation fuel identification, building/residential natural gas detection, formaldehyde detection, smoke detection, fire detection, automatic ventilation control applications (cooking, smoking, etc.), air intake monitoring, hospital/medical anesthesia & sterilization gas detection, infectious disease detection and breath applications, body fluids analysis, pharmaceutical applications, drug discovery, telesurgery, and the like.

This application discloses several numerical range limitations that support any range within the disclosed numerical ranges even though a precise range limitation is not stated verbatim in the specification because the embodiments of the invention could be practiced throughout the disclosed numerical ranges. Finally, the entire disclosure of the patents and publications referred in this application, if any, are hereby incorporated herein in entirety by reference.

1. A method of fabricating a surface enhanced fluorescent (SEF) nanoparticle comprising:
obtaining one or more core nanoparticles with desired plasmonic properties; and
adding a fluorophore zone on the one or more core nanoparticles.

2. The method of claim 1 wherein the method further comprises coating the core nanoparticle with a spacer layer.

3. The method of claim 2 wherein the method further comprises coating the spacer layer with a primer layer.

4. The method of claim 3 wherein the method further comprises coating the primer layer or the spacer later with an encapsulation layer.

5. The method of claim 1 wherein the fluorophore zone is coated directly on the one or more core nanoparticles.

6. The method of claim 1 wherein the core nanoparticle is a metal.

7. The method of claim 6 wherein the core metal nanoparticle comprises an alloy.

8. The method of claim 7 wherein the core metal nanoparticle comprises a noble metal.

9. The method of claim 8, wherein the core metal nanoparticle comprises a metal selected from the group consisting of Au, Ag, Cu, Al, Pd, Ni, Pt, and alloys thereof.

10. The method of claim 6, wherein the core metal nanoparticle comprises Au or an Au alloy.

11. The method of claim 1 wherein the spacer layer comprises silica or an organic polymer.

12. The method of claim 1 wherein the spacer layer comprises silica.

13. The method of claim 1 wherein the optional encapsulation layer comprises silica or an organic layer.

14. The method of claim 1 wherein the SEF nanoparticle comprises 1 core metal nanoparticle.

15. The method of claim 1 wherein the SEF nanoparticle comprises 2 or more core metal nanoparticles.

16. A method comprising:
attaching one or more affinity agents to one or more SEF nanoparticles, wherein the SEF nanoparticles comprise one or more core nanoparticles and a fluorophore zone;
contacting the SEF nanoparticle to at least one target analyte; and
detecting the SEF nanoparticle to detect the at least one target analyte.

17. The method of claim 16, wherein the affinity agent is selected from the group consisting of an antibody, antigen, ligand, receptor, aptamer, nucleic acid, protein, peptide, and carbohydrate.

18. The method of claim 16, wherein the affinity agent comprises an antibody.

19. The method of claim 16, wherein the affinity agent comprises a nucleic acid.

20. The method of claim 16, wherein the target analyte comprises a biomolecule.

21. The method of claim 16, wherein the target analyte comprises a biological species.

22. The method of claim 16, wherein the target analyte comprises a pathogen.

23. The method of claim 16, wherein the target analyte comprises an infectious agent.

24. The method of claim 16, wherein the target analyte comprises a disease cell.

25. The method of claim 16, wherein the target analyte comprises an organism.

26. The method of claim 16, wherein the target analyte comprises a tissue sample.

27. The method of claim 16, wherein the target analyte comprises a nucleic acid.

28. The method of claim 16, wherein the target analyte comprises a protein.

29. A SEF nanoparticle comprising:
one or more core nanoparticles; and
a fluorophore zone.

30. The SEF nanoparticle of claim 29 further comprising a spacer layer located between the core nanoparticle and the fluorophore zone.

31. The SEF nanoparticle of claim 29 further comprising a primer layer.

32. The SEF nanoparticle of claim 29 further comprising an encapsulation layer surrounding SEF nanoparticle.

33. The SEF nanoparticle of claim 29 wherein the core nanoparticle comprises a metal.

34. The SEF nanoparticle of claim 33 wherein the core nanoparticle comprises a noble metal.

35. The SEF nanoparticle of claim 34 wherein the core nanoparticle comprises an alloy.

36. The SEF nanoparticle of claim 34, wherein the core nanoparticle comprises a metal selected from the group consisting of Au, Ag, Cu, Al, Pd, Pt, and alloys thereof.

37. The SEF nanoparticle of claim 34, wherein the core nanoparticle comprises Ag, Au or alloys thereof.

38. The SEF nanoparticle of claim 30 wherein the spacer layer comprises silica or organic polymer.

39. The SEF nanoparticle of claim 38 wherein the spacer layer comprises silica.

40. The SEF nanoparticle of claim 32 where in the encapsulation layer comprises silica or an organic layer.

41. The SEF nanoparticle of claim 29 wherein the core nanoparticle comprises 1 metal nanoparticle.

42. The SEF nanoparticle of claim 29 wherein the core nanoparticle comprises2 or more metal nanoparticles.

43. The SEF nanoparticle of claim 29 further comprising at least one affinity agent attached to the SEF nanoparticle.

44. The SEF nanoparticle of claim 43, wherein the affinity agent is selected from the group consisting of an antibody, antigen, ligand, receptor, aptamer, and nucleic acid.

45. The SEF nanoparticle of claim 43, wherein the affinity agent is an antibody.

46. The SEF nanoparticle of claim 43, wherein the affinity agent comprises a nucleic acid.

47. A kit comprising:
one or more reagents comprising core nanoparticles;
one or more optional reagents for adding a spacer layer;
one or more optional reagents for adding a primer layer;
one or more reagents for adding a fluorophore zone; and
one or more optional reagents for adding an ecapsulation layer.

48. The kit of claim 43 wherein the core nanoparticles are metal nanoparticles.

49. The kit of claim 48 wherein the core metal nanoparticles are alloys.

50. The kit of claim 48 wherein the core metal nanoparticles are noble metals or alloys thereof.

51. The kit of claim 47 wherein the one or more optional reagents for the spacer layer comprise silica or organic polymer.

52. The kit of claim 47 wherein the one or more reagents for the fluorophore layer comprise porous silica or an organic polymer matrix.

53. The kit of claim 47 further comprising reagents for attaching one or more affinity agents to the SEF nanoparticle.

54. The kit of claim 53, wherein the affinity agent is an antibody.

55. The kit of claim 53, wherein the affinity agent comprises nucleic acid.

56. A kit comprising one or more SEF nanoparticles and one or more affinity agents, wherein the SEF nanoparticles comprise one or more core nanoparticles and a fluorophore zone.

57. The kit of claim 56 wherein the core nanoparticles comprise a metal.

58. The kit of claim 57 wherein the metal comprises a noble metal.

59. The kit of claim 56 wherein the SEF nanoparticle further comprises a spacer layer and an encapsulation layer.
